(19) United States
(12) Patent Application Publication
(10) Pub. No.: US 2003/0149384 A1
(43) Pub. Date: Aug. 7, 2003

(54) Forced ventilated anatomical supports

(76) Inventors: Leonard G. Davis, Newport Beach, CA (US); Lytton A. Williams, Rolling Hills Estate, CA (US); Sui-Kay Wong, Hong Kong (CN)

Correspondence Address: OPPENHEIMER WOLFF & DONNELLY LLP, 840 NEWPORT CENTER DRIVE, SUITE 700, NEWPORT BEACH, CA, 92660, US

(21) Appl. No.: 10/221,763

(22) PCT Filed: Mar. 16, 2001
(86) PCT No.: PCT/US01/40318

(51) Int. Cl. A61F 5/00

(52) U.S. Cl. 602/14

(57) ABSTRACT

An anatomical support (1) for immobilizing or supporting a body portion fitted with a fan (9) for providing controlled airflow in a channel (8) that is located in the side of the anatomical support (1) approximate to the body portion of the user and/or between the anatomical support (1) and the body portion of the user.

### FIELD OF THE INVENTION

The present invention relates generally to an anatomical support for fully or partially immobilizing a body portion and more particularly pertains to a new ventilated anatomical support having controlled airflow between the anatomical support and the corresponding body portion of the user.

### BACKGROUND OF THE INVENTION

The present invention is in the field of medical devices, namely anatomical supports, braces or casts, such as cervical collars, lumbar braces, wrist, arm, neck and leg casts and braces. The present invention is equally applicable to other protective devices used adjacent to body parts such as, for example, protective helmets.

A commonly used prior art anatomical brace, the Thomas Collar, consists of a foam member that is wrapped around the neck to anatomically support the neck and head of the user and relieve pressure on the cervical portion of the spine. The Thomas Collar is typically used following an injury, such as, for example, whiplash, sporting injury, or surgery upon the cervical portion of the spine. Similar prior art anatomical supports are also used around the lower vertebrae proximate the lumbar region of the back. Additionally, as will be understood by those of ordinary skill in the art, a large variety of anatomical supports, braces and casts are used on numerous other body parts in order to facilitate the healing of, or to otherwise protect the, corresponding body area. Such typical prior art supports, braces and casts are used on, for example, human knees, ankles, feet, legs, humorous, ulna, wrist, and hand. Of course, such supports, braces and casts are not limited to use on humans, but may be used on animals in veterinary practices as well including, for example, horses, bovine, canines, and the like.

Because an enclosed space having minimal, if any, air flow, is formed between the anatomical support and the body portion, heat and moisture typically build up between prior art anatomical supports and the corresponding body portion of the user. Prior art anatomical supports generally become uncomfortable to wear and can result in an increased likelihood of infections and, in the event of open wounds, increased duration of time required for healing. Furthermore, the discomfort associated with prior art supports increases the likelihood that the user will not wear the support against a physician's instructions. An additional problem associated with currently available anatomical supports is that they are not easily placed in the proper position resulting in insufficient, less efficient, or improper support.

The new, unique and useful aspects of the present invention overcome these and other shortcomings of current anatomical supports.

### SUMMARY OF THE PRESENT INVENTION

The present invention relates to a novel anatomical support which preferably employs a powered forced air flow device, such as, for example, a fan, blower, or air compressor to provide airflow between the anatomical support and the corresponding portion of the body of the user in order to provide airflow therebetween and/or to decrease the temperature and humidity between the anatomical support and the corresponding body part. The powered forced air flow device can be either attached directly to the support or connected to an opening in the support by a hose, tube, or other suitable mechanism. As will be appreciated by those of ordinary skill in the art, and persons involved in the provision of health care, the present invention not only substantially enhances the comfort associated with utilizing anatomical supports, and potentially increases the likelihood that a user will comply with a physician's instructions to wear the support, but it also facilitates the healing of wounds, if any, which may be located within the region of the anatomical support.

The present invention also preferably provides a new and unique manner in which to regulate and control the temperature and humidity of the air gap in the area between an anatomical support and the corresponding portion of the body of the user by preferably employing a controlled air flow device which may preferably be intermittently activated or, alternatively, has variable speed capabilities to increase or decrease the amount of air flow. One preferred embodiment of the controlled air flow device also incorporates the use of: (i) a sensor to monitor the corresponding temperature between the anatomical support and the corresponding body part; (ii) a sensor to monitor the corresponding humidity between anatomical support and the corresponding body part; (iii) a manual air flow control which can be adjusted by the user; and (iv) a microprocessor to adjust the controlled air flow device in response to input from the temperature sensor and humidity sensor. As persons of ordinary skill in the art will appreciate, the present invention may incorporate any one or a combination of any of these devices.

The present invention preferably employs a fan, such as a battery-powered propeller fan, to cause air to flow into, out of, or through a space or channel formed between the corresponding body portion of the user and the anatomical support, or between the body of the user and the anatomical support. The fan may also be powered by other means such as, for example, alternating current, a generator, or solar power source.

The anatomical support is preferably comprised of two or more support members, which when joined or connected together preferably form a rigid support generally conformed in contour to the corresponding body portion to be immobilized or supported and, when joined or connected, preferably results in the arrangement of at least one communicating and corresponding interior channels in the interior surface of the respective anatomical support members proximate the corresponding body portion of the user and/or between the body of the user and the interior surface of the corresponding anatomical support members. Similar to the Thomas Collar, the anatomical support of the present invention may also be comprised of a single rigid member having first and second ends, such as foam, which is wrapped around the corresponding body portion and then the first and second ends are connected. One or more of the members of the support of the present invention preferably possess one or more openings in the exterior surface of the support members which are employed and configured either to receive and house a fan or to connect to a remote forced air flow device through flexible air hose or tube. The support members of the present invention may accommodate one or a plurality of fans, depending upon the body portion to be supported, the size of the fan employed, the volume of air desired to be moved, and the target temperature and humidity to be achieved.

The interior surface of the support member preferably has an open channel through which air may easily flow. In one preferred embodiment of the present invention the interior surface of the support members are preformed such that they have a predefined open channel running from end to end. In another preferred embodiment, where a soft material, such as foam, is connected to the interior surface of the support members, the foam is either preferably connected to only portions of the interior surface of the support members such that an open channel is thereby formed where the foam is not affixed or, alternatively, the foam preferably has a varied thickness resulting in the thinner thicknesses of the foam creating a channel. When the support members of the present invention are utilized and placed in position by a user, the one or more interior channels facilitate the movement of air within the neck brace by allowing air generated by a fan to flow over at the immobilized body part. In yet another embodiment of the present invention, the soft material, such as foam, used on the interior surface of the support members may have a relatively uniform thickness and, instead of creating one or more distinct channels created by decreased thickness or voids of material, the interior surface preferably possesses indentations, grooves, or the like, to allow for the creation of one or more channels through which air may travel. Preferably, the channels of the present invention allow air to flow unencumbered, or controlled, around the neck of the user.

The fan of the present invention is preferably powered by a low direct current voltage source, preferably below 36 volts. In one preferred embodiment, three volts is sufficient, derived from two standard AA batteries. However, other battery and energy options and sources may also be used including, for example, nickel cadmium, lithium ion, solar, or other known sources. Because the amperage of some preferred embodiments of the fan is so low, the preferred battery sources will last for long periods of time without requiring recharging or replacement. Additionally, because in some preferred embodiments the fan does not provide a cooling effect, but rather serves to remove air from the channel and replace it with fresh air, the speed of the fan and volume of air flow may be relatively low and the fan will preferably remain relatively quiet. Furthermore, because air is not forced into the channel in some preferred embodiments, but is preferably pulled and removed from the channel, foreign particles from the air are preferably not forced into an area that may have open wounds. In another preferred embodiment, air may be forced into the channel via the fan, however a filter is used to remove impurities or particles from the air prior to being transferred into the channel adjacent the corresponding body part.

In a preferred embodiment the power supply will be built into the compartment holding the fan so that no extension cord or separate battery compartment will be necessary. Further, the fan and/or battery compartment may preferably be detachable from the anatomical support. The fan and/or battery compartment can be held in place by a snap fit, or other suitable means of holding it in place, including a separate strap arrangement surrounding the brace.

### BRIEF DESCRIPTION OF THE DRAWINGS

FIG. 1 is a front view of the exterior surface of one preferred embodiment of the present invention;

FIG. 2 is a left perspective view of one preferred embodiment of the present invention;.

FIG. 3 is a top view of one preferred embodiment of the present invention;

FIG. 4 is a bottom left perspective view of one preferred embodiment of the present invention;

FIG. 5 is a front view of the exterior surface of one preferred embodiment of the present invention;

FIG. 6 is a right perspective view of one embodiment of the present invention;

FIG. 7 is a rear view of the outside surface of the back support member of one embodiment of the present invention;

FIG. 8 is the front view of the interior surface of the back support member of one embodiment of the present invention;

FIG. 9 is the back view of the interior surface of the front support member of one embodiment of the present invention; and

FIG. 10 is a right sectional view of one preferred embodiment with the cloth cover removed.

### DETAILED DESCRIPTION OF THE INVENTION

In the present disclosure, for ease of description, reference is made to several preferred embodiments relating to a cervical anatomical support used around the neck of a user. However, the present invention, as will be appreciated by those skilled in the art, is equally applicable to any type of anatomical support, brace and cast, and, of course, for all other appropriate parts of an anatomical structure, human or otherwise. Additionally, the presently described invention may also be used in protective devices such as, for example, helmets including motorcycle, bicycle, football, hockey, and other recreational and sporting helmets and devices.

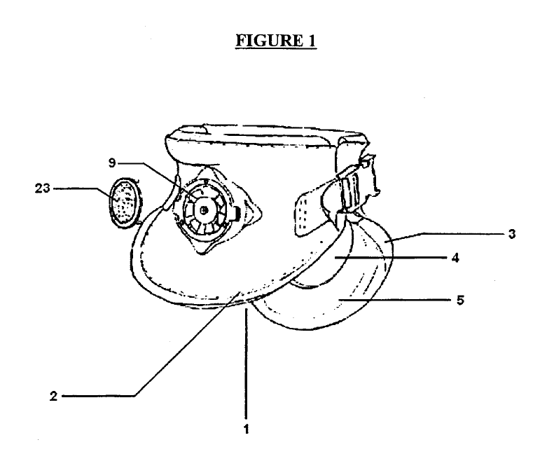

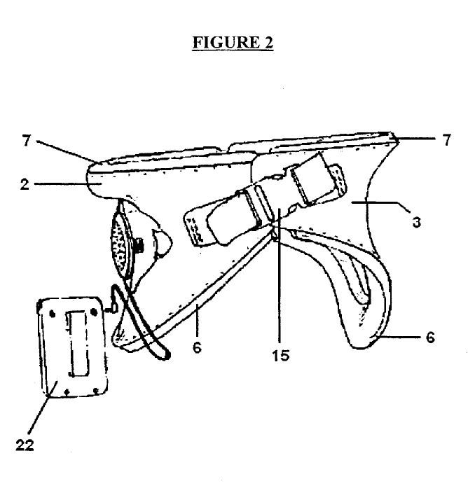

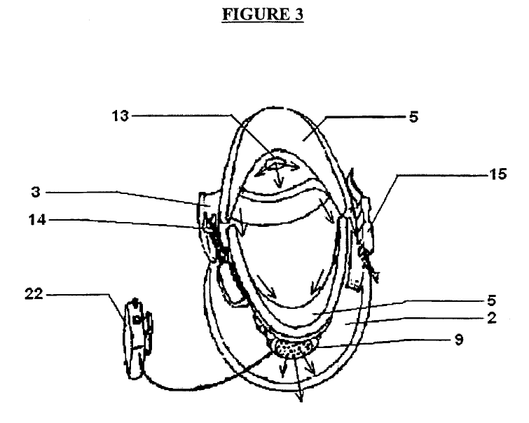

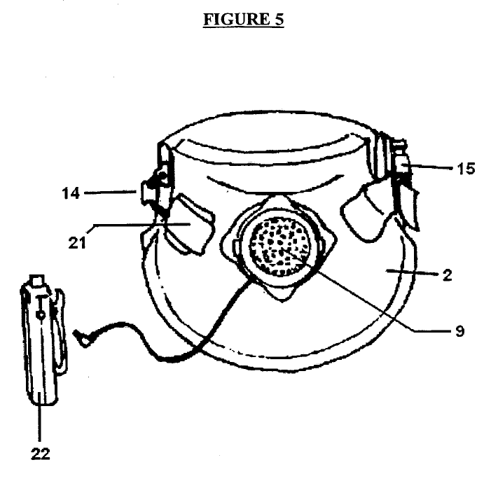

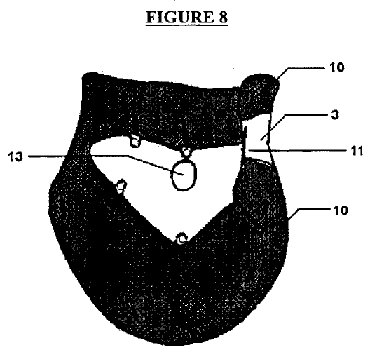

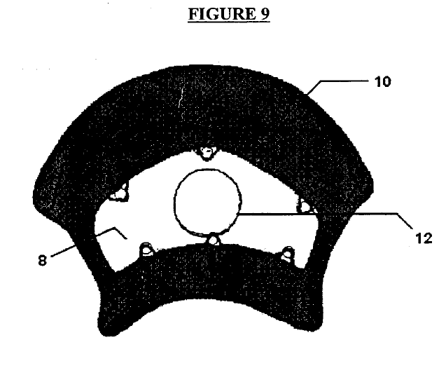

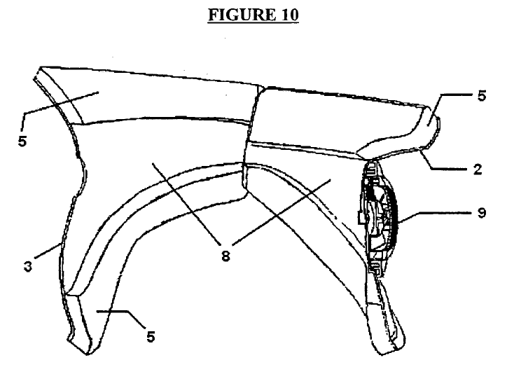

As exhibited in FIGS. 1-10, a preferred embodiment of a neck brace employing the present invention comprises two separate members, a front support member 2 and back support member 3, forming two halves of an anatomical support 1. The support members 2 and 3 are preferably made from a firm and light weight material such as, for example, pp (poly-propane) medical grade PVC. Of course, persons of ordinary skill in the art will recognize that numerous types of materials and composite, such as graphite or foam, would be suitable for this purpose. The interior surface 4 of each support member 2 and 3 preferably has a soft surface or, in some preferred embodiments, if the material which comprises support members 2 and 3 is not sufficiently soft, the interior surface 14 of the support members may preferably be covered by a soft material 5 such as, for example, foam, or other soft substance. The bottom and top edges 6 and 7 of the support members are similarly covered with a soft substance. The soft material is attached using means which are well known in the field such as adhesives, glue, Velcro®, loop and button, or other well known connection methods.

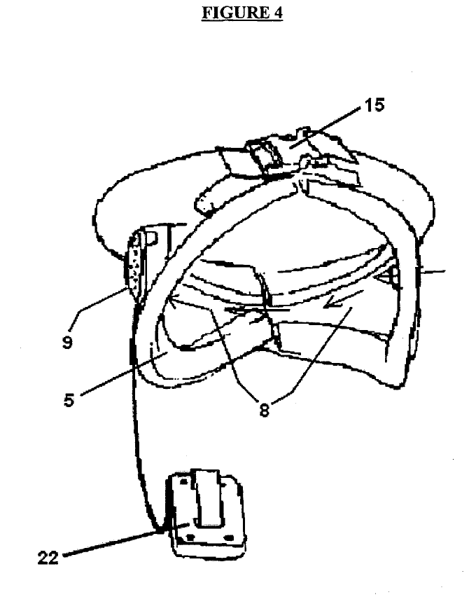

The interior surface 4 of the support members 2 and 3 preferably have an open channel 8 through which air may easily flow. In one preferred embodiment of the present invention, the interior surface 4 of the support members 2 and 3 are preformed such that they have a predefined open channel 8 running from one end of each support member to the other end of the support member. In another preferred embodiment, where a soft material 5 is adjacent to the interior surface 4 of the support members 2 and 3, the soft material 5 is either preferably connected to only portions of the interior surface 4 of the support members 2 and 3, as shown in FIG. 4, such that an open channel 4 is thereby formed where the soft material 5 is not affixed or, alternatively, the soft material 5 preferably has a varied thickness resulting in the thinner thicknesses of the foam creating a channel 8. When the support members 2 and 3 of the present invention are utilized and placed in position by a user, the one or more interior channels 8 facilitate the movement of air within the neck brace by allowing air generated by a fan 9 to flow over at the immobilized body part. In yet another embodiment of the present invention, the soft material 5 used on the interior surface 4 of the support members 2 and 3 may have a relatively uniform thickness and, instead of creating one or more distinct channels 8 created by decreased thickness or voids of material, the interior surface preferably possesses indentations, grooves, or the like, to allow for the creation of one or more conduits through which air may travel. Preferably, the channels 8 of the present invention allow air to flow unencumbered, or controlled, around the corresponding body part of the user.

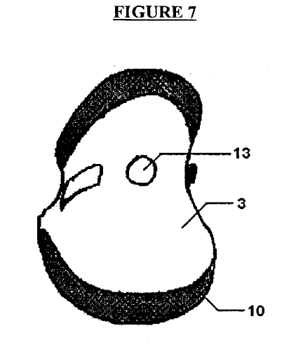

In one preferred embodiment, the soft material 5 employed on the interior surface 4 of the support members 2 and 3 is covered by a second preferably porous material, such as, preferably, 100 percent cotton cloth 10. The cloth material 10 preferably covers the soft material 5 and is attached to the support members 2 and 3, as shown in FIGS. 7-9, by wrapping over and partially covering the exterior surface 11 of the support members 2 and 3. Of course, the cloth material 10 may be affixed or connected to the support members 2 and 3 in numerous ways well known in the art such as, for example, tie strings, Velcro®, elastic bands, and other well known methods. The material, such as cloth, is preferably removable and washable since this is the portion of the anatomical support that will likely, over time, become soiled as it is exposed to the surface of the skin of the user.

As shown in FIGS. 1-6, the present invention preferably employs a fan 9, such as a battery-powered propeller fan, to cause air to flow into, out of, or through a channel 8 formed between the corresponding body portion of the user and the anatomical support 1, or between the body of the user and the anatomical support 1. The fan 9 may also be powered by other means such as, for example, alternating current, a generator, or solar power source. The anatomical support 1 is preferably comprised of two or more support members 2 and 3, which when joined or connected together preferably form a rigid anatomical support 1 generally conformed in contour to the corresponding body portion to be immobilized or supported and, when joined or connected, preferably results in the arrangement of at least one communicating and corresponding interior channels 8 in the interior surface 4 of the respective anatomical support members 2 and 3 proximate the corresponding body portion of the user and/or between the body of the user and the interior surface 4 of the corresponding anatomical support members 2 and 3.

In another embodiment of the present invention, the anatomical support 1 may be comprised of a single rigid member having first and second ends which is wrapped around the corresponding body portion and then the first and second ends are connected. If desired, the anatomical support 1 of the present invention may be made with any number of support members.

One or more of the support members 2 and 3 of the anatomical support 1 of the present invention preferably possess one or more openings 12 in the exterior surface 11 of the support members 2 and 3 which are employed and configured to receive and house a fan 9. The support members 2 and 3 of the present invention may accommodate one or a plurality of fans 9, depending upon the body portion to be supported, the size of the fan 9 employed, the volume of air desired to be moved, and the target temperature and humidity to be achieved.

As shown in FIGS. 1-6, preferably, one or more fans 9 are connected to or within the corresponding openings 12 in the exterior surface 11 of the support members 2 and 3. The openings 10 preferably directly communicate with, or have conduits which communicate with, the channel 8 formed between the interior surface 15 of the support members 2 and 3 and the corresponding body part. Furthermore, alternative embodiments of the present invention preferably have one or more additional openings 13 in the exterior surface 11 of the support members 2 and 3 which likewise communicate with the interior channel 8 thus permitting air flow in or out of said channels 8. However, in some preferred embodiments, such additional openings 13 are not accompanied by fans, rather, they are employed for the purpose of facilitating greater air flow and ventilation through the channel.

In one preferred embodiment of the present invention a battery-powered fan 9, such as a propeller fan, is employed to preferably pull or force air to flow out of the channel 12 formed on the interior surface 4 of the support members 2 and 3 proximate the body portion of the user and/or between the body of the user and the anatomical support 1 and through the fan 9.

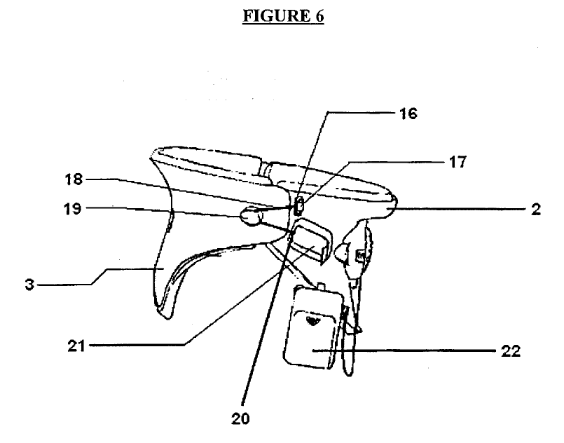

One end of each respective support member 2 and 3 are preferably affixed to one another in a manner such that they may pivot with respect to one another. Any appropriate means may be used for this purpose, such as, for example, butt hinges, piano hinges, flexible materials (including plastic, string or leather), or any other material or mechanism generally known or used in the art. In the preferred embodiment shown in FIG. 6, one end of each support member 2 and 3 are pivotally connected to each other by employing a pulley arrangement 14 and by a snap belt arrangement 15 on the opposite end. In the pulley arrangement 14, the first end of a cord 16 is fixed to the upper exterior surface of the front support member 2 of the neck brace by a cord retainer 17, the cord 18 passes around a pulley 19 on the side of the back support member 3 of the neck brace. The second end of the cord 20 is attached to a second cord retainer 21. This pulley cord system enables the tightening force to double the pull force exert to the cord by the user and makes it easier for the user to tighten up the front and back support members. A wide variety of conventional fasteners may be employed including the commercially available fabric attachment fastener sold under the trademark VELCRO®.

After being affixed to one another, the two halves (support members 2 and 3) of the anatomical support 1 are preferably opened wide enough to receive the corresponding anatomical portion of the user, such as a neck, and, after receiving the body portion, the two support members 2 and 3 are then closed so that the respective remaining ends of the support members 2 and 3 may be connected to complete and close the brace, cast or support member around the body part. The user may preferably attach the remaining respective ends of the support members to one another by well known means such as, for example, Velcro®, hook and loop attachments, tie strings, or any other well known methods.

As shown in FIGS. 1-6 in one preferred embodiment of the invention, the user first attaches the snap belt arrangement 15, or other suitable attachment for causing the two support members 1 and 2 to pivot for receiving the neck of the user. The two support members 1 and 2 of the neck brace are then fitted around the neck and closed so that the bottom edge 7 rests comfortably on the shoulders of the user. The user then attaches the other side of the neck brace by means of the pulley attachment 14 so that it is held snugly, but comfortably, in place.

One or more openings 12 in the exterior surface 11 of the support members 2 and 3 of the anatomical support 1 are preferably fitted with a small detachable battery-operated fan 9 that, when activated, causes air to flow through the channel 8 located in the interior surface 4 of the anatomical support 1 proximate the body portion of the user and/or between the body of the user and the anatomical support 1. Preferably, the fan operates so that air is drawn or removed from the channel. Of course, in another embodiment the flow of air may preferably be reversed to also allow for air to be forced into the channel 8. Additionally, there can be one or more additional openings 13 in the support members 2 and 3 of the anatomical support 1 which communicate with the channel 8 to which fans 9 are not affixed but through which air flow is further facilitated.

As shown in FIGS. 1-6, a fan 9 may preferably interference or snap fit into an opening 12 in the front support member 2 of the neck brace after the neck brace is in place. It may also be placed into the opening 12 prior to placing the neck brace on the user. Other means of attaching the fan 9 to the opening 12 may also be used. The fan 9 is preferably removably detachable from the opening 12 in the front of the neck brace so that it may be services or replaced. Retractable snap clips are used in the preferred embodiment, although other means of attaching the fan assembly 9, may be used.

The fan of the present invention is preferably powered by a low direct current voltage source, preferably below 36 volts. In one preferred embodiment the power source is combined with the fan. However, a separate battery pack 22 may also be used. The power supply preferably has a control for turning the power on and off and for preferably controlling the speed of the fan 9. Also, an intermittent or variable fan control may be employed for intermittently automatically turning the fan 9 on and off to save battery power or regulate temperature, humidity or other air circulation variables. Once the anatomical support 1 is in place, the power is turned on activating one or more fans 9. The user, based on the user's preferences and personal comfort, can manually control the speed of the fan 9. Alternatively, a microprocessor may be employed so that the fan 9 control and regulation may be preprogrammed to achieve the desired characteristics. When activated, the fan draws air from inside the channel 8 created between the support members 2 and 3 and the neck of the user. Fresh air is preferably drawn through the additional opening 13 and out the opening 12 in which the fan 9 is housed. In such configuration an optional filter may cover the additional opening 13 to help prevent foreign particles and impurities from entering the channel 8. Alternatively, the fan 9 may force air into the channel 8 and out through the additional opening 13. In this configuration an optional filter 23 may be employed with the fan 9 to help prevent foreign particles and impurities from entering the channel 8.

In one preferred embodiment the power supply will be built into the compartment holding the fan so that no extension cord or separate battery compartment will be necessary. Further, the fan and/or battery compartment may preferably be detachable from the anatomical support. The fan and/or battery compartment can be held in place by a snap fit, or other suitable means of holding it in place, including a separate strap arrangement surrounding the brace. In one preferred embodiment, three volts of power is sufficient, being the size of two standard AA batteries. However, other battery and energy options and sources may also be used including, for example, nickel cadmium, lithium ion, solar, or other known sources may also be used. Because the amperage of some preferred embodiments of the fan is so low, the preferred battery sources will last for long periods of time without requiring recharging or replacement.

We claim:

1. An anatomical support for immobilizing a body portion comprising:
one or more support members, having an interior surface and an exterior surface and conforming in contour to the body portion to be immobilized such that at least one channel is formed between the body portion and the interior surface;
at least one of the support members having at least one opening through the exterior surface;
at least one air flow device coupled to the at least one opening through the exterior surface to generate air flow though said channel.

2. The anatomical support of claim 1, wherein at least one support member has a second opening through the exterior surface to improve the flow of air through said channel.

3. The anatomical support of claim 1, wherein the one or more support members comprise
a plurality of support members, the anatomical support further comprising at least one releasable mechanism for securing said members in joined relation to each other.

4. The anatomical support of claim 3 further comprising at least one hinge mechanism for further securing said support members in joined relation.

5. The anatomical support of claim 3 wherein the one or more support members comprise a first support member and second support member and wherein the at least one releasable mechanism comprises:
a first cord retainer for fixing a first end of a cord to the first support member;
a pulley attached to the second support member;
a cord that extends from the first cord retainer and around the pulley; and
a second end of the cord that is attached to a second cord retainer on the first support member.

6. The anatomical support of claim 5 wherein the first cord retainer or second cord retainer comprise a fabric attachment mechanism.

7. The anatomical support of claim 1 wherein the at least one air flow device is detachably removable from the opening.

8. The anatomical support of claim 1 wherein the at least one air flow device provides intermittent air flow.

9. The anatomical support of claim 1 wherein the at least one air flow device is controlled by a sensor to monitor the corresponding temperature of the area located between the anatomical support and the corresponding body portion.

10. The anatomical support of claim 1 wherein the at least one air flow device is controlled by a sensor to monitor the corresponding humidity of the area located between the anatomical support and the corresponding body portion.

11. The anatomical support of claim 1 wherein the at least one air flow device is manually controlled.

12. The anatomical support of claim 1 wherein the at least one air flow device is controlled by a microprocessor.

13. The anatomical support of claim 1 wherein the at least one air flow device is electrically-powered.

14. The anatomical support of claim 1 wherein the at least one air flow device is battery-powered.

15. The anatomical support of claim 14 further comprising a battery compartment that is separate from said air flow device.

16. The anatomical support of claim 1 wherein the at least one air flow device is a fan.

17. The anatomical support of claim 1 wherein the air flow device is connected to said opening with a hose.

18. An anatomical support for immobilizing a body portion, wherein the support comprises:
separate front and rear support members, each having an interior surface and an exterior surface and conforming in contour to the body portion to be immobilized such that at least one channel is formed between the body portion and the interior surface;
at least one of the support members having at least one opening on the exterior surface;
at least one air flow device connected to at least one corresponding opening on the exterior surface to generate air flow through said channel; and
at least one releasable mechanism for securing said support members in joined relation.

19. The anatomical support of claim 18, wherein the at least one support member has at least one second opening to facilitate a flow of air in said channel.

20. The anatomical support of claim 18 further comprising at least one hinge mechanism for further securing said members in joined relation.

21. The anatomical support of claim 18 where in the at least one releasable mechanism comprises:
a first cord retainer for fixing a first end of a cord to the rear support member;
a pulley attached to the front support member;
a cord that extends from the cord retainer and around the pulley; and
a second end of the cord that is attached to a second cord retainer on the rear support member.

22. The anatomical support of claim 21 wherein the first cord retainer or second cord retainer comprises a fabric attachment mechanism.

23. The anatomical support of claim 18 where in the at least one releasable mechanism comprises:
a first cord retainer for fixing a first end of a cord to the front support member;
a pulley attached to the rear support member;
a cord that extends from the first cord retainer and around the pulley; and
a second end of the cord that is attached to a second cord retainer on the rear support member.

24. The anatomical support of claim 23 wherein the at least one cord retainer comprises a fabric attachment mechanism.

25. The anatomical support of claim 18 wherein the at least one air flow device is detachably removable from the opening.

26. The anatomical support of claim 18 wherein the at least one air flow device provides intermittent air flow.

27. The anatomical support of claim 18 wherein the at least one air flow device is controlled by a sensor to monitor the corresponding temperature of the area located between the anatomical support and the corresponding body portion.

28. The anatomical support of claim 18 wherein the at least one air flow device is controlled by a sensor to monitor the corresponding humidity of the area located between the anatomical support and the corresponding body portion.

29. The anatomical support of claim 18 wherein the at least one air flow device is manually controlled.

30. The anatomical support of claim 18 wherein the at least one air flow device is controlled by a microprocessor.

31. The anatomical support of claim 18 wherein the at least one air flow device is electrically-powered.

32. The anatomical support of claim 18 wherein the at least one air flow device is battery-powered.

33. The anatomical support of claim 32 further comprising a battery compartment that is separate from said air flow device.

34. The anatomical support of claim 18 wherein the at least one opening through the exterior surface is covered with a filter.

35. The anatomical support of claim 18 wherein at least one air flow device is covered with a filter.

36. The anatomical support of claim 18 further comprising a foam material attached to the interior surface wherein the foam material possess indentations creating the at least one channel.

37. A protective helmet for protecting the head of the user comprising:
at least one member, having an interior surface and an exterior surface and conforming in contour to the head of the user such that at least one channel is formed between the head of the user and the interior surface;
at least one of the members having at least one opening on the exterior surface; and
at least one air flow device connected to at least one corresponding opening on the exterior surface to generate air flow through said channel.

38. An anatomical support for immobilizing the neck of a user comprising:
separate front and rear support members, having an interior surface and an exterior surface, conforming in contour to the body portion to be immobilized;
wherein the interior surface of the front and rear support members are at least partially covered by a foam material, which is covered by a washable cloth material, such that a channel is formed between the body of the user and the interior surface that lacks the foam material;
at least one support member having at least one opening through the exterior surface;
at least one battery-powered fan coupled to at least one opening through the exterior surface to facilitate the removal of air from said channel;
at least one member having a second opening to further facilitate the flow of air in said channel; and
two releasable mechanisms for securing said members in joined relation to each other.
